(19) United States
(12) Patent Application Publication
(10) Pub. No.: US 2020/0042036 A1
(43) Pub. Date: Feb. 6, 2020

(54) Wearable Device with Automatic and Temporary Sensor-Activated Band Contraction

(71) Applicant: Robert A. Connor, St. Paul, MN (US)

(72) Inventor: Robert A. Connor, St. Paul, MN (US)

(73) Assignee: Medibotics LLC, St. Paul, MN (US)

(21) Appl. No.: 16/598,514

(22) Filed: Oct. 10, 2019

Related U.S. Application Data
(63) Continuation-in-part of application No. 16/568,580, filed on Sep. 12, 2019.
Application No. 16/568,580 is a continuation-in-part of application No. 15/294,746, filed on Oct. 16, 2016.
Application No. 15/294,746 is a continuation-in-part of application No. 15/963,061, filed on Apr. 25, 2018.
Application No. 15/963,061 is a continuation-in-part of application No. 15/725,330, filed on Oct. 5, 2017.
Application No. 15/725,330 is a continuation-in-part of application No. 15/431,769, filed on Feb. 14, 2017.
Application No. 15/431,769 is a continuation-in-part of application No. 15/418,620, filed on Jan. 27, 2017.
Application No. 15/418,620 is a continuation-in-part of application No. 15/294,746, filed on Oct. 16, 2016.
Application No. 15/963,061 is a continuation-in-part of application No. 14/550,953, filed on Nov. 22, 2014, now abandoned.
Application No. 14/550,953 is a continuation-in-part of application No. 15/431,769, filed on Feb. 14, 2017.
Application No. 15/431,769 is a continuation-in-part of application No. 14/951,475, filed on Nov. 24, 2015, now Pat. No. 10,314,492.
Application No. 14/951,475 is a continuation-in-part of application No. 15/294,746, filed on Oct. 16, 2016.
Application No. 15/294,746 is a continuation-in-part of application No. 15/206,215, filed on Jul. 8, 2016, now abandoned.
Application No. 15/206,215 is a continuation-in-part of application No. 14/951,475, filed on Nov. 24, 2015, now Pat. No. 10,314,492.
Application No. 14/951,475 is a continuation-in-part of application No. 14/951,475, filed on Nov. 24, 2015, now Pat. No. 10,314,492.
Application No. 14/951,475 is a continuation-in-part of application No. 14/623,337, filed on Feb. 16, 2015, now Pat. No. 9,582,035.
Application No. 14/623,337 is a continuation-in-part of application No. 14/951,475, filed on Nov. 24, 2015, now Pat. No. 10,314,492.
Application No. 14/951,475 is a continuation-in-part of application No. 14/623,337, filed on Feb. 16, 2015, now Pat. No. 9,582,035.
Application No. 14/623,337 is a continuation-in-part of application No. 14/071,112, filed on Nov. 4, 2013, now abandoned.
Application No. 14/071,112 is a continuation-in-part of application No. 13/901,131, filed on May 23, 2013, now Pat. No. 9,536,449.
(60) Provisional application No. 62/857,942, filed on Jun. 6, 2019.
(60) Provisional application No. 62/814,692, filed on Mar. 6, 2019.
(60) Provisional application No. 62/814,713, filed on Mar. 6, 2019.
(60) Provisional application No. 62/857,942, filed on Jun. 6, 2019.
(60) Provisional application No. 62/814,692, filed on Mar. 6, 2019.
(60) Provisional application No. 62/814,713, filed on Mar. 6, 2019.
(60) Provisional application No. 62/549,587, filed on Aug. 24, 2017.
(60) Provisional application No. 62/439,147, filed on Dec. 26, 2016.
(60) Provisional application No. 62/439,147, filed on Dec. 26, 2016.
(60) Provisional application No. 62/349,277, filed on Jun. 13, 2016.
(60) Provisional application No. 62/311,462, filed on Mar. 22, 2016.
(60) Provisional application No. 62/439,147, filed on Dec. 26, 2016.
(60) Provisional application No. 62/297,827, filed on Feb. 20, 2016.
(60) Provisional application No. 62/349,277, filed on Jun. 13, 2016.
(60) Provisional application No. 62/245,311, filed on Oct. 23, 2015.
(60) Provisional application No. 62/349,277, filed on Jun. 13, 2016.
(60) Provisional application No. 62/245,311, filed on Oct. 23, 2015.
(60) Provisional application No. 62/115,691, filed on Feb. 13, 2015.
(60) Provisional application No. 62/113,423, filed on Feb. 7, 2015.
(60) Provisional application No. 62/111,163, filed on Feb. 3, 2015.
(60) Provisional application No. 62/106,856, filed on Jan. 23, 2015.
(60) Provisional application No. 62/100,217, filed on Jan. 6, 2015.
(60) Provisional application No. 61/948,124, filed on Mar. 5, 2014.
(60) Provisional application No. 61/944,090, filed on Feb. 25, 2014.

(51) Int. Cl. G06F 1/16; A61B 5/00

(52) U.S. Cl. CPC G06F 1/163; A61B 2562/0247; A61B 2562/0219; A61B 5/681

(57) ABSTRACT

This invention is wearable computing device for measuring biometric parameters whose contraction (e.g. tightness or looseness) around a person's wrist and/or forearm is automatically and temporarily changed based on information from one or more motion, optical, and/or electromagnetic energy sensors.

### CROSS-REFERENCE TO RELATED APPLICATIONS

This application is a continuation in part of U.S. patent application Ser. No. 16/568,580 filed on 2019 Sep. 12. This application claims the priority benefit of U.S. provisional patent application 62/857,942 filed on 2019 Jun. 6. This application claims the priority benefit of U.S. provisional patent application 62/814,692 filed on 2019 Mar. 6. This application claims the priority benefit of U.S. provisional patent application 62/814,713 filed on 2019 Mar. 6. This application is a continuation in part of U.S. patent application Ser. No. 15/294,746 filed on 2016 Oct. 16.

U.S. application Ser. No. 16/568,580 claimed the priority benefit of U.S. provisional patent application 62/857,942 filed on 2019 Jun. 6. U.S. application Ser. No. 16/568,580 claimed the priority benefit of U.S. provisional patent application 62/814,692 filed on 2019 Mar. 6. U.S. application Ser. No. 16/568,580 claimed the priority benefit of U.S. provisional patent application 62/814,713 filed on 2019 Mar. 6. U.S. application Ser. No. 16/568,580 was a continuation in part of U.S. patent application Ser. No. 15/963,061 filed on 2018 Apr. 25. U.S. application Ser. No. 16/568,580 was a continuation in part of U.S. patent application Ser. No. 15/725,330 filed on 2017 Oct. 5. U.S. application Ser. No. 16/568,580 was a continuation in part of U.S. patent application Ser. No. 15/431,769 filed on 2017 Feb. 14. U.S. application Ser. No. 16/568,580 was a continuation in part of U.S. patent application Ser. No. 15/418,620 filed on 2017 Jan. 27. U.S. application Ser. No. 16/568,580 was a continuation in part of U.S. patent application Ser. No. 15/294,746 filed on 2016 Oct. 16.

U.S. application Ser. No. 15/963,061 was a continuation in part of U.S. patent application Ser. No. 14/550,953 filed on 2014 Nov. 22.

U.S. application Ser. No. 15/725,330 claimed the priority benefit of U.S. provisional patent application 62/549,587 filed on 2017 Aug. 24. U.S. application Ser. No. 15/725,330 was a continuation in part of U.S. patent application Ser. No. 15/431,769 filed on 2017 Feb. 14. U.S. application Ser. No. 15/725,330 claimed the priority benefit of U.S. provisional patent application 62/439,147 filed on 2016 Dec. 26. U.S. application Ser. No. 15/725,330 was a continuation in part of U.S. patent application Ser. No. 14/951,475 filed on 2015 Nov. 24 which issued as U.S. patent Ser. No. 10/314,492 on 2019 Jun. 11.

U.S. application Ser. No. 15/431,769 claimed the priority benefit of U.S. provisional patent application 62/439,147 filed on 2016 Dec. 26. U.S. application Ser. No. 15/431,769 was a continuation in part of U.S. patent application Ser. No. 15/294,746 filed on 2016 Oct. 16. U.S. application Ser. No. 15/431,769 was a continuation in part of U.S. patent application Ser. No. 15/206,215 filed on 2016 Jul. 8. U.S. application Ser. No. 15/431,769 claimed the priority benefit of U.S. provisional patent application 62/349,277 filed on 2016 Jun. 13. U.S. application Ser. No. 15/431,769 claimed the priority benefit of U.S. provisional patent application 62/311,462 filed on 2016 Mar. 22.

U.S. application Ser. No. 15/418,620 claimed the priority benefit of U.S. provisional patent application 62/439,147 filed on 2016 Dec. 26. U.S. application Ser. No. 15/418,620 claimed the priority benefit of U.S. provisional patent application 62/297,827 filed on 2016 Feb. 20. U.S. application Ser. No. 15/418,620 was a continuation in part of U.S. patent application Ser. No. 14/951,475 filed on 2015 Nov. 24 which issued as U.S. patent Ser. No. 10/314,492 on 2019 Jun. 11.

U.S. application Ser. No. 15/294,746 claimed the priority benefit of U.S. provisional patent application 62/349,277 filed on 2016 Jun. 13. U.S. application Ser. No. 15/294,746 was a continuation in part of U.S. patent application Ser. No. 14/951,475 filed on 2015 Nov. 24 which issued as U.S. patent Ser. No. 10/314,492 on 2019 Jun. 11. U.S. application Ser. No. 15/294,746 claimed the priority benefit of U.S. provisional patent application 62/245,311 filed on 2015 Oct. 23. U.S. application Ser. No. 15/294,746 was a continuation in part of U.S. patent application Ser. No. 14/623,337 filed on 2015 Feb. 16 which issued as U.S. Pat. No. 9,582,035 on 2017 Feb. 28.

U.S. application Ser. No. 15/206,215 claimed the priority benefit of U.S. provisional patent application 62/349,277 filed on 2016 Jun. 13. U.S. application Ser. No. 15/206,215 was a continuation in part of U.S. patent application Ser. No. 14/951,475 filed on 2015 Nov. 24 which issued as U.S. patent Ser. No. 10/314,492 on 2019 Jun. 11.

U.S. application Ser. No. 14/951,475 claimed the priority benefit of U.S. provisional patent application 62/245,311 filed on 2015 Oct. 23. U.S. application Ser. No. 14/951,475 was a continuation in part of U.S. patent application Ser. No. 14/623,337 filed on 2015 Feb. 16 which issued as U.S. Pat. No. 9,582,035 on 2017 Feb. 28. U.S. application Ser. No. 14/951,475 was a continuation in part of U.S. patent application Ser. No. 14/071,112 filed on 2013 Nov. 4. U.S. application Ser. No. 14/951,475 was a continuation in part of U.S. patent application Ser. No. 13/901,131 filed on 2013 May 23 which issued as U.S. Pat. No. 9,536,449 on 2017 Jan. 3.

U.S. application Ser. No. 14/623,337 claimed the priority benefit of U.S. provisional patent application 62/115,691 filed on 2015 Feb. 13. U.S. application Ser. No. 14/623,337 claimed the priority benefit of U.S. provisional patent application 62/113,423 filed on 2015 Feb. 7. U.S. application Ser. No. 14/623,337 claimed the priority benefit of U.S. provisional patent application 62/111,163 filed on 2015 Feb. 3. U.S. application Ser. No. 14/623,337 claimed the priority benefit of U.S. provisional patent application 62/106,856 filed on 2015 Jan. 23. U.S. application Ser. No. 14/623,337 claimed the priority benefit of U.S. provisional patent application 62/100,217 filed on 2015 Jan. 6. U.S. application Ser. No. 14/623,337 claimed the priority benefit of U.S. provisional patent application 61/948,124 filed on 2014 Mar. 5. U.S. application Ser. No. 14/623,337 claimed the priority benefit of U.S. provisional patent application 61/944,090 filed on 2014 Feb. 25.

The entire contents of these related applications are incorporated herein by reference.

### FEDERALLY SPONSORED RESEARCH

Not Applicable

### SEQUENCE LISTING OR PROGRAM

Not Applicable

### BACKGROUND

### Field of Invention

This invention relates to wearable biometric measurement devices.

### Introduction

There are many potential health-related benefits from non-invasive measurement of biometric parameters such as oxygenation level, hydration level, glucose level, pulse rate, heart rate variability, and blood pressure. Considerable progress has been made in recent years toward measuring many of these parameters by incorporating biometric sensors into wearable devices such as smart watches and bands. However, technological challenges remain. For example, measurement of biometric parameters via wearable devices can be hindered by movement of a device relative to a person's body, especially when a person is being very physically active. For example, a smart watch or band can shift on a person's wrist as a person moves, changing the distances between one or more biometric sensors and body tissue, thereby hindering accurate measurement of biometric parameters. This can be addressed by keeping the smart watch or band very tight all the time, but this can be uncomfortable and potentially unhealthy. A better solution is needed.

### Review of the Relevant Art

U.S. application publication 20170119314 (Just et al., May 4, 2017, “Physiological Monitoring Devices with Adjustable Stability”) discloses a wearable monitoring device with a sensor and an actuator that is triggered by the sensor to change the stability of the device. U.S. application publications 20160255944 (Baranski et al., Sep. 8, 2016, “Dynamic Fit Adjustment for Wearable Electronic Devices”), 20180027931 (Baranski et al., Feb. 1, 2018, “Dynamic Fit Adjustment for Wearable Electronic Devices”), 20190082800 (Baranski et al., Mar. 21, 2019, “Dynamic Fit Adjustment for Wearable Electronic Devices”), and also U.S. Pat. No. 9,781,984 (Baranski et al., Oct. 10, 2017, “Dynamic Fit Adjustment for Wearable Electronic Devices”), Ser. No. 10/149,521 (Baranski et al., Dec. 11, 2018, “Dynamic Fit Adjustment for Wearable Electronic Devices”), and Ser. No. 10/398,200 (Baranski et al., Sep. 3, 2019, “Dynamic Fit Adjustment for Wearable Electronic Devices”) disclose systems and methods which use tensioner to adjust the fit of a wearable electronic. U.S. application publication 20170086743 (Bushnell et al., Mar. 30, 2017, “Sensing Contact Force Related to User Wearing an Electronic Device”) discloses using a force sensor to determine the tightness of a wearable band.

U.S. Pat. No. 8,522,456 (Beers et al., Sep. 3, 2013, “Automatic Lacing System”), U.S. Pat. No. 8,769,844 (Beers et al., Jul. 8, 2014, “Automatic Lacing System”) and U.S. Pat. No. 9,307,804 (Beers et al., Apr. 12, 2016, “Automatic Lacing System”) disclose footwear with an automatic lacing system. U.S. application publications 20140070042 (Beers et al., Mar. 13, 2014, “Motorized Tensioning System with Sensors”) and 20160272458 (Beers et al., Sep. 22, 2016, “Motorized Tensioning System with Sensors”), as well as U.S. Pat. No. 9,365,387 (Beers et al., Jun. 14, 2016, “Motorized Tensioning System with Sensors”) disclose footwear with a tensioning member that is tightened or loosened using a motorized tensioning device for winding and unwinding the tensioning member on a spool. U.S. application publication 20160345653 (Beers et al., Dec. 1, 2016, “Lockout Feature for a Control Device”) discloses footwear with a lockout feature to be used in conjunction with a motorized tensioning system. U.S. application publication 20160345661 (Beers et al., Dec. 1, 2016, “Sole Plate for an Article of Footwear”) discloses footwear with a sole plate can include one or more specialized compartments which house a motorized tensioning system.

U.S. Pat. No. 9,204,690 (Alt et al., Dec. 8, 2015, “Device for Automatically Tightening and Loosening Shoe Laces”) discloses a device for automatically tying and untying shoe laces. U.S. Pat. No. 7,752,774 (Ussher, Jul. 13, 2010, “Powered Shoe Tightening with Lace Cord Guiding System”) discloses an automatic shoe lace tightening or power lace system. U.S. application publication 20080086911 (Labbe, Apr. 17, 2008, “Weight-Activated Tying Shoe”) discloses a weight-activated tying shoe.

U.S. Pat. No. 6,491,647 (Bridger et al., Dec. 10, 2002, “Physiological Sensing Device”) discloses a non-invasive device which quantifies displacement, force, motion, vibration and acoustic effects from biological functions. U.S. application publication 20080060224 (Whittlesey et al., Mar. 13, 2008, “Shoe with Sensors, Controller and Active-Response Elements and Method for Use Thereof”) discloses golf shoes whose characteristics are changed based on the wearer's activity. U.S. Pat. No. 5,509,423 (Bryars, Apr. 23, 1996, “Pump Band”) discloses a wrist-worn pneumatic pump assembly. U.S. Pat. No. 9,144,168 (Sedillo et al., Sep. 22, 2015, “Appendage-Mounted Display Apparatus”) discloses a wearable cuff with a ratcheting lacing or strap system. U.S. application publication 20140094728 (Soderberg et al., Apr. 3, 2014, “Motorized Tensioning System for Medical Braces and Devices”) discloses a spool-based tensioning system for medical devices. U.S. application publication 20170034615 (Mankodi et al., Feb. 2, 2017, “Integration of Sensors into Earphones”) discloses an earphone with an optical couple which fits into a wearer's ear. U.S. application publication 20160287108 (Wei et al., Oct. 6, 2016, “Light Guide System for Physiological Sensor”) discloses a physiological sensor with a light guide system. U.S. Pat. No. 6,251,080 (Henkin et al., Jun. 26, 2001, “Self Contained Ambulatory Blood Pressure Cincture”) discloses an ambulatory inflatable arm band for measuring blood pressure.

U.S. Pat. No. 5,485,848 (Jackson et al., Jan. 23, 1996, “Portable Blood Pressure Measuring Device and Method of Measuring Blood Pressure”) discloses a portable device for monitoring a user's arterial blood pressure. U.S. Pat. No. 6,151,968 (Chou, Nov. 28, 2000, “Wrist Band Type Electronic Sphygmomanometer”) discloses a wrist-worn electronic sphygmomanometer. U.S. Pat. No. 6,799,887 (Kinney, Oct. 5, 2004, “Wristwatch Guard with Access Flap”) discloses a flap to cover and protect a wristwatch. U.S. application publication 20090253996 (Lee et al., Oct. 8, 2009, “Integrated Sensor Headset”) discloses a head-worn device for measuring physiological data. U.S. application publication 20040025984 (Holemans et al., Feb. 12, 2004, “Jewelry Arrangements”) discloses a piece of jewelry with shape memory material. U.S. Pat. No. 9,129,503 (Borlenghi, Sep. 8, 2015, “Locking GPS Device for Locating Children”) discloses a GPS-based child-locator device which can be securely locked to a child's wrist or ankle. U.S. application publication 20070125816 (Myers, Jun. 7, 2007, “Locking Mechanism for Use with Ratchet or Cog Strap”) discloses a locking mechanism for use with ratchet or cog strap. U.S. application publication 20150065891 (Wiesel, Mar. 5, 2015, “Method and Apparatus for Detecting Atrial Fibrillation”) discloses a method of detecting atrial fibrillation.

U.S. application publication 20120102691 (Han et al., May 3, 2012, “Strap Connecting Member and Electronic Device with the Strap Connecting Member”) and U.S. Pat. No. 8,370,998 (Han et al., Feb. 12, 2013, “Strap Connecting Member and Electronic Device with the Strap Connecting Member”) disclose a device strap which is automatically shrunk using a torsion spring. U.S. patent Ser. No. 10/082,872 (Cruz-Hernandez et al., Sep. 25, 2018, “Deformable Haptic Wearables with Variable Physical Properties”) discloses a haptically-enabled device with a physical property such as length, stiffness, or texture which is changed. U.S. application publication 20130053661 (Alberth et al., Feb. 28, 2013, “System for Enabling Reliable Skin Contract of an Electrical Wearable Device”) discloses the use of thermocouples to adjust a wearable band. U.S. Pat. No. 8,147,417 (Gavriely, Apr. 3, 2012, “Tourniquet Timer”) discloses a tourniquet with a timer.

U.S. Pat. No. 9,471,102 (Townsend et al., Oct. 18, 2016, “Connection by Securing Device with Integrated Circuitry Layer”) and U.S. application publication 20160324470 (Townsend et al., Nov. 10, 2016, “Novel Securing Device for Connecting to a Display Device”) disclose a wearable device with circuitry in both a display and a band. U.S. application publication 20100234714 (Mercier et al., Sep. 16, 2010, “Dynamic Body State Display Device”) discloses a flexible body state display device. U.S. Pat. No. 6,301,754 (Grunberger et al., Oct. 16, 2001, “Magnetic Closure Device for Clothing Items, Leather Goods and the Like”) discloses a magnetic closure device for clothing or accessories. U.S. Pat. No. 7,620,450 (Kim et al., Nov. 17, 2009, “Method and System for Removing Noise by Using Change in Activity Pattern”) discloses activity-specific methods for biometric signal noise removal. U.S. application publication 20070244399 (Kim et al., Oct. 18, 2007, “Pulse Measurement Device, Method and Medium”) discloses the combined use of a photoplethysmography (PPG) sensor and acceleration sensor. U.S. Pat. No. 6,461,125 (Terasawa et al., Oct. 8, 2002, “Air Pump, Air Chamber Device Using Air Pump, and Wristwatch Having Air Chamber Device”) discloses an air chamber device. U.S. Pat. No. 6,648,502 (Oomori et al., Nov. 18, 2003, “Wrist-Portable Electronic Apparatus and Air Chamber Device”) discloses a wrist-worn with a flexible expansion chamber.

U.S. application publication 20080167572 (Stivoric et al., Jul. 10, 2008, “Systems, Methods, and Devices to Determine and Predict Physilogical States of Individuals and to Administer Therapy, Reports, Notifications, and the Like Therefor”) discloses systems and methods for predicting physiological and conditional states and events based on sensed data. U.S. Pat. No. 9,044,372 (Wild et al., Jun. 2, 2015, “Compression Device for the Limb”) discloses a compression device for a patient's limb. U.S. Pat. No. 4,331,154 (Broadwater et al., May 25, 1982, “Blood Pressure and Heart Rate Measuring Watch”) discloses a watch with a piezoelectric transducer which measures blood pressure and heart rate. U.S. Pat. No. 4,941,236 (Sherman et al., Jul. 17, 1990, “Magnetic Clasp for Wristwatch Strap”) discloses a watch including an overlapping strap with magnetizable material. U.S. Pat. No. 7,690,220 (Okamura, Apr. 6, 2010, “Personal Ornament”) discloses a personal ornament with strong metallic magnets. U.S. Pat. No. 7,618,384 (Nardi et al., Nov. 17, 2009, “Compression Device, System and Method of Use”) discloses an apparatus and method for cyclically compressing the limb of a patient.

U.S. application publication 20150135410 (Wu et al., May 21, 2015, “Belt Structure”) and U.S. Pat. No. 9,144,273 (Wu et al., Sep. 29, 2015, “Belt Structure”) discloses a belt with shape memory alloys. U.S. patent Ser. No. 10/143,271 (Zhang et al., Dec. 4, 2018, “Smart Watch and Automatic Wearing Method Thereof”) discloses a smart watch with controlled extension or retraction of sections of the watch band. U.S. Pat. No. 7,562,640 (Lalor, Jul. 21, 2009, “Animal Collar”) discloses an electronic animal collar which reduced the load on the animal's neck by one or more stimulating electrodes.

### SUMMARY OF THE INVENTION

This invention is a wearable computing device for the wrist and/or forearm comprising: a display; a sensor; and an attachment member which is configured to be fastened around the wrist and/or forearm, wherein the device has a first configuration in which the attachment member has a first degree of contraction in order to fit in a relatively loose manner around the wrist and/or forearm and a second configuration in which the attachment member has a second degree of contraction in order to fit in a relatively snug manner around the wrist and/or forearm, and wherein the device automatically transitions from the first configuration to the second configuration based on information from the sensor.

The attachment member can be a (watch) strap, band, bracelet, ring, armlet, cuff, or sleeve. The sensor can be a motion sensor, optical sensor, or (other) electromagnetic energy sensor. The attachment member can be automatically and temporarily contracted when data from the sensor indicates that the device is not able to measure biometric parameters accurately because the device is moving relative to the person's body. For example, the attachment member can be automatically and temporarily contracted with the person is being very physically active. In an example, the attachment member can be made with material which contracts when exposed to electrical current. In an example, the attachment member can have one or more contracting actuators which respond to data from a sensor. This invention can address the problem of inaccurate biometric measurement from a wearable device due to movement.

### BRIEF DESCRIPTION OF THE FIGURES

FIG. 1 shows a wearable computing device for the wrist and/or arm comprising a display member, a sensor, and a sensor-activated contracting attachment member.

### DETAILED DESCRIPTION OF THE FIGURES

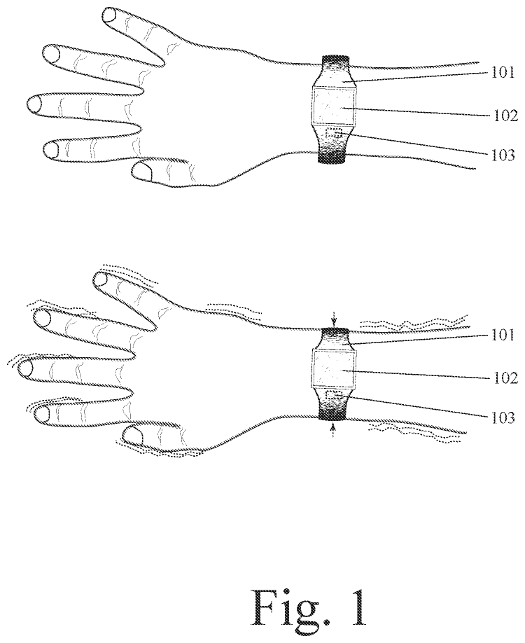

FIG. 1 shows an example of a wearable computing device for the wrist and/or arm comprising: a display member 102; a sensor 103; and a contracting attachment member 101. This device has a first configuration in which the contracting attachment member has a first degree of contraction and a second configuration in which the contracting attachment member has a second degree of contraction. In FIG. 1, the upper portion shows this device in the first configuration and the lower portion shows this device in the second configuration.

The device automatically transitions from the first configuration to the second configuration based on information from the sensor. In this example, the sensor is a motion sensor and the contracting attachment member automatically contracts when the sensor detects a high level of movement. This can enable the device to fit in a relatively loose manner when the person is relatively stationary and to fit in a relatively snug manner when the person is relatively active. This can help to make the device fit comfortably when the person is resting, but not slip off when the person is very active. In an example, the display can be selected from the group consisting of: a computer display screen, a semi-transparent display, and a projected image.

In an example, an attachment member can be a (watch) strap, band, bracelet, ring, armlet, cuff, or sleeve. In an example, an attachment member can be attached to a person's wrist and/or forearm by connecting two ends of the attachment member with a clasp, clip, buckle, hook, pin, plug, or hook-and-eye mechanism. In an example, an attachment member can have buckles, snaps, adhesive, hook-and-eye mechanisms or other connecting elements so as to be fastened around the circumference of the wrist and/or forearm. In an example, an attachment member can be a band with a buckle, clasp, clip, hook, hook-and-eye material, pin, latch, button, or zipper which fastens around the perimeter of a person's wrist and/or forearm.

In an example, an attachment member can be attached to a person's wrist and/or forearm by stretching and sliding it over the person's hand onto the wrist and/or forearm. In an example, an attachment member can be stretched or expanded around the hand to slip onto the wrist and/or forearm. In an example, an attachment member can be stretchable, expandable, and/or elastic so that it can be slipped over a person's hand onto their wrist and/or forearm. In an example, an attachment member can span between 50% and 95% of the circumference of the wrist and/or forearm and be flexible enough to bend around the wrist and/or forearm. In an example, the attachment member can be attached to a person's wrist and/or forearm by applying force to pull two ends apart to slip the member over the wrist and/or forearm, wherein the two ends retract back towards each other when the force is removed.

In an example, a sensor can be a motion sensor. In an example, a sensor can be an accelerometer, gyroscope, inclinometer, and/or compass. In an example, a sensor can be an optical sensor. In an example, a sensor can be an optoelectronic sensor, infrared light sensor, ultraviolet light sensor, spectroscopic sensor, and/or other light-spectrum-analyzing sensor. In an example, a spectroscopic sensor can collect data concerning the spectrum of light reflected from and/or transmitted through tissue of the person's wrist and/or forearm. In an example, a sensor can be a piezoelectric sensor, pressure sensor, and/or strain gauge. In an example, a sensor can be a blood pressure sensor, electrocardiogram (ECG) sensor, glucose sensor, heart rate sensor, and/or blood oximetry sensor. In an example, a sensor can be an electromagnetic energy sensor. In an example, a sensor can measure the resistance, impedance, and/or conductivity of tissue of the person's wrist and/or forearm with respect to the transmission of electromagnetic energy. In an example, a sensor can measure electromagnetic energy emitted from muscles and/or nerves in the person's wrist and/or forearm. In an example, the sensor can be a capacitive electromagnetic energy sensor.

In an example, a device worn on a person's wrist and/or forearm can have sensors at different circumferential locations around a person's wrist and/or forearm. In an example, a device can have a two-dimensional array of sensors. Sensors in this two-dimensional array can differ in location circumferentially (at different locations around the circumference of the device) and/or laterally (at different locations along axes which are perpendicular to the circumference of the device). In an example, a two-dimensional sensor array can be part of the circumference-center-facing surface of an enclosure which is on the anterior (upper) portion of a device. In an example, a two-dimensional sensor array can be on the circumference-center-facing surface of a band or strap.

Having a circumferentially-distributed array of sensors can allow a wearable device to record biometric measurements from different locations along the circumference of a person's wrist and/or forearm. This can help to find the best location on a person's wrist and/or forearm from which to most-accurately record biometric measurements. Having a circumferentially-distributed array of sensors can also enable a device to record biometric measurements from substantially the same location on a person's wrist and/or forearm, even if the device is unintentionally slid, shifted, and/or partially-rotated around the person's wrist and/or forearm. A different primary sensor can selected to record data when a device slides, shifts, and/or rotates. This can help to reduce biometric measurement errors when a device is slid, shifted, and/or partially-rotated around a person's wrist and/or forearm.

In an example, an attachment member can be made with material which contracts when exposed to electrical current. In an example, an attachment member can have one or more actuators which respond to data from a sensor. In an example, an attachment member can be contracted and/or shortened when information from a sensor on the device indicates that the sensor is not able to make accurate biometric measurements. In an example, an attachment member can be contracted and/or shortened when information from a sensor on the device indicates that the sensor is too far away from the surface of the person's wrist and/or forearm and cannot make accurate biometric measurements. In an example, an attachment member can be contracted and/or shortened when information from a sensor on the device indicates that the sensor is too far away from the surface of the person's wrist and/or forearm to make accurate biometric measurements because the device has shifted relative to the person's wrist and/or forearm. In an example, an attachment member can be contracted and/or shortened when information from a sensor on the device indicates that the sensor moving too much relative to surface of the person's wrist and/or forearm and cannot make accurate biometric measurements.

In an example, an attachment member can be contracted and/or shortened when information from an optical (e.g. spectroscopic) sensor on the device indicates that the optical (e.g. spectroscopic) sensor is not able to make accurate biometric measurements. In an example, an attachment member can be contracted and/or shortened when information from a optical (e.g. spectroscopic) sensor on the device indicates that the optical (e.g. spectroscopic) sensor is too far away from the surface of the person's wrist and/or forearm to make accurate biometric measurements. In an example, an attachment member can be contracted and/or shortened when information from a optical (e.g. spectroscopic) sensor on the device indicates that the optical (e.g. spectroscopic) sensor is too far away from the surface of the person's wrist and/or forearm to make accurate biometric measurements because the device has shifted relative to the person's wrist and/or forearm. In an example, an attachment member can be contracted and/or shortened when information from an optical (e.g. spectroscopic) sensor on the device indicates that the optical (e.g. spectroscopic) sensor moving too much relative to surface of the person's wrist and/or forearm and cannot make accurate biometric measurements.

In an example, an attachment member can be contracted and/or shortened when information from an electromagnetic energy sensor on the device indicates that the electromagnetic energy sensor is not able to make accurate biometric measurements. In an example, an attachment member can be contracted and/or shortened when information from an electromagnetic energy sensor on the device indicates that the electromagnetic energy sensor is too far away from the surface of the person's wrist and/or forearm to make accurate biometric measurements. In an example, an attachment member can be contracted and/or shortened when information from an electromagnetic energy sensor on the device indicates that the electromagnetic energy sensor is too far away from the surface of the person's wrist and/or forearm to make accurate biometric measurements because the device has shifted relative to the person's wrist and/or forearm. In an example, an attachment member can be contracted and/or shortened when information from an electromagnetic energy sensor on the device indicates that the electromagnetic energy sensor moving too much relative to surface of the person's wrist and/or forearm and cannot make accurate biometric measurements.

In an example, a wearable computing device for the wrist and/or forearm can comprise: a display; a motion sensor; and an attachment member which is configured to be fastened around the wrist and/or forearm, wherein the device has a first configuration in which the attachment member has a first degree of contraction in order to fit in a relatively loose manner around the wrist and/or forearm and a second configuration in which the attachment member has a second degree of contraction in order to fit in a relatively snug manner around the wrist and/or forearm, and wherein the device transitions from the first configuration to the second configuration based on information from the motion sensor. In an example, the motion sensor can include one or more components selected from the group consisting of: accelerometer; gyroscope; and inclinometer. In an example, the attachment member can contract it is when exposed to electrical current. In an example, the device can further comprise an electromagnetic actuator which contracts the attachment member.

In an example, a wearable computing device for the wrist and/or forearm can comprise: a display; a optical sensor; and an attachment member which is configured to be fastened around the wrist and/or forearm, wherein the device has a first configuration in which the attachment member has a first degree of contraction in order to fit in a relatively loose manner around the wrist and/or forearm and a second configuration in which the attachment member has a second degree of contraction in order to fit in a relatively snug manner around the wrist and/or forearm, and wherein the device transitions from the first configuration to the second configuration based on information from the optical sensor. In an example, the optical sensor can include one or more components selected from the group consisting of: spectroscopic sensor, light-spectrum-analyzing sensor, optoelectronic sensor, PPG sensor, and infrared light sensor. In an example, the attachment member can contract it is when exposed to electrical current. In an example, the device can further comprise an electromagnetic actuator which contracts the attachment member.

In an example, a wearable computing device for the wrist and/or forearm can comprise: a display; a compression sensor; and an attachment member which is configured to be fastened around the wrist and/or forearm, wherein the device has a first configuration in which the attachment member has a first degree of contraction in order to fit in a relatively loose manner around the wrist and/or forearm and a second configuration in which the attachment member has a second degree of contraction in order to fit in a relatively snug manner around the wrist and/or forearm, and wherein the device transitions from the first configuration to the second configuration based on information from the compression sensor. In an example, the compression sensor can include one or more components selected from the group consisting of: pressure sensor, strain gauge, and piezoelectric sensor. In an example, the attachment member can contract it is when exposed to electrical current. In an example, the device can further comprise an electromagnetic actuator which contracts the attachment member.

In an example, a wearable computing device for the wrist and/or forearm can comprise: a display; a motion sensor; and an attachment member which is configured to be fastened around the wrist and/or forearm, wherein the device has a first configuration in which the attachment member has a first length in order to fit in a relatively loose manner around the wrist and/or forearm and a second configuration in which the attachment member has a second length in order to fit in a relatively snug manner around the wrist and/or forearm, and wherein the device transitions from the first configuration to the second configuration based on information from the motion sensor. In an example, the motion sensor can include one or more components selected from the group consisting of: accelerometer; gyroscope; and inclinometer. In an example, the attachment member can contract it is when exposed to electrical current. In an example, the device can further comprise an electromagnetic actuator which contracts the attachment member.

In an example, a wearable computing device for the wrist and/or forearm can comprise: a display; a optical sensor; and an attachment member which is configured to be fastened around the wrist and/or forearm, wherein the device has a first configuration in which the attachment member has a first length in order to fit in a relatively loose manner around the wrist and/or forearm and a second configuration in which the attachment member has a second length in order to fit in a relatively snug manner around the wrist and/or forearm, and wherein the device transitions from the first configuration to the second configuration based on information from the optical sensor. In an example, the optical sensor can include one or more components selected from the group consisting of: spectroscopic sensor, light-spectrum-analyzing sensor, optoelectronic sensor, PPG sensor, and infrared light sensor. In an example, the attachment member can contract it is when exposed to electrical current. In an example, the device can further comprise an electromagnetic actuator which contracts the attachment member.

In an example, a wearable computing device for the wrist and/or forearm can comprise: a display; a compression sensor; and an attachment member which is configured to be fastened around the wrist and/or forearm, wherein the device has a first configuration in which the attachment member has a first length in order to fit in a relatively loose manner around the wrist and/or forearm and a second configuration in which the attachment member has a second length in order to fit in a relatively snug manner around the wrist and/or forearm, and wherein the device transitions from the first configuration to the second configuration based on information from the compression sensor. In an example, the compression sensor can include one or more components selected from the group consisting of: pressure sensor, strain gauge, and piezoelectric sensor. In an example, the attachment member can contract it is when exposed to electrical current. In an example, the device can further comprise an electromagnetic actuator which contracts the attachment member.

In an example, an array of sensors can comprise an arcuate array of light emitters and light receivers which is distributed around the circumference of a device which is worn around a person's wrist, finger, or arm. With an arcuate array of light emitters and light receivers around the circumference of a person's wrist, finger, or arm—at least some of the light emitters and light receivers will be in close optical communication with the person's body tissue even if the device shifts and/or rotates around the wrist, finger, or arm. Also, if one specific area of the circumference of the wrist, finger, or arm is particularly good for measuring a particular biometric parameter (e.g. next to a specific vascular structure), then at least some of the light emitters and light receivers will be in close optical communication with that specific area even if the device shifts and/or rotates. Further, having a plurality of light emitters and light receivers around the circumference of a person's wrist, finger, or arm can provide spectroscopic scanning of a specific region of body tissue from different angles and/or using different wavelengths. Spectroscopic scanning of the same specific region from different angles and/or using different wavelengths can provide multivariate information for more accurate measurement of elusive analytes such as blood glucose.

In an example, an array of sensors can comprise an array of one or more light emitters and one or more light receivers which are configured to be worn around a person's wrist, arm, or finger. Light energy from a light emitter can be received by a light receiver after the light has been transmitted through or reflected from the person's body tissue. In an example, changes in the spectrum of the light energy caused by transmission of the light energy through the person's body tissue and/or reflection of the light energy from the person's body tissue can be analyzed in order to measure one or more biometric parameters concerning the person.

In an example, changes in the spectrum of the light energy caused by transmission of the light energy through the person's body tissue and/or reflection of the light energy from the person's body tissue can be analyzed in order to measure the person's oxygenation level. In an example, changes in the spectrum of the light energy caused by transmission of the light energy through the person's body tissue and/or reflection of the light energy from the person's body tissue can be analyzed in order to measure the person's hydration level. In an example, changes in the spectrum of the light energy caused by transmission of the light energy through the person's body tissue and/or reflection of the light energy from the person's body tissue can be analyzed in order to measure the person's glucose level.

In an example, changes in the intensity and/or spectrum of the light energy caused by transmission of the light energy through the person's body tissue and/or reflection of the light energy from the person's body tissue can be analyzed in order to measure the person's pulse rate. In an example, changes in the intensity and/or spectrum of the light energy caused by transmission of the light energy through the person's body tissue and/or reflection of the light energy from the person's body tissue can be analyzed in order to measure the person's heart rate variability. In an example, changes in the intensity and/or spectrum of the light energy caused by transmission of the light energy through the person's body tissue and/or reflection of the light energy from the person's body tissue can be analyzed in order to measure the person's blood pressure. In an example, an array of sensors can comprise an array of PPG (photoplethysmography) sensors.

In an example, an array of light emitters and light receivers can collectively span at least half of the circumference of a person's wrist, arm, or finger. In an example, an array of light emitters and light receivers can collectively span at least three-quarters of the circumference of a person's wrist, arm, or finger. In an example, light emitters in an array can collectively span at least half of the circumference of a person's wrist, arm, or finger and light receivers in the array can also collectively span at least half of the circumference of the person's wrist, arm, or finger. In an example, light emitters in an array can collectively span at least three-quarters of the circumference of a person's wrist, arm, or finger and light receivers in the array can also collectively span at least three-quarters of the circumference of the person's wrist, arm, or finger.

In an example, there can be an alternating sequence of light emitters and light receivers around a circumferential line around the circumference of the device and/or around (at least half of) the circumference of a person's wrist, finger, or arm. In an example, there can be a repeating sequence of light emitters and light receivers around a circumferential line around (at least half of) the circumference of the device and/or around the circumference of a person's wrist, finger, or arm. In an example, there can be proximal pairs of light emitters and light receivers. In an example, an array can be a circumferential array of pairs of light emitters and light receivers. In an example, a paired light emitter and light receiver can both be located on a line which is orthogonal to a circumference of the device.

In an example, there can be proximal triads of light emitters and light receivers. In an example, an array can be a circumferential array of triads of light emitters and light receivers. In an example, an array can be a circumferential array of triads, each of which comprises one light emitter and two light receivers. In an example, an array can be a circumferential array of triads, each of which comprises two light emitters and one light receiver. In an example, an array can include a series of proximal sets of light emitters and light receivers. In an example, each proximal set can comprise one light emitter and a plurality of proximal light receivers. In an example, each proximal set can comprise a plurality of light receivers and one light emitter. In an example, each proximal set can comprise one light emitter and a plurality of proximal light receivers in a ring (or polygonal) configuration around the light emitter. In an example, each proximal set can comprise one light receiver and a plurality of proximal light emitters in a ring (or polygonal) configuration around the light receiver.

In an example, a first light emitter in an array can be located in a first quadrant of the circumference of the person's wrist, arm, or finger; a second light emitter in the array can be located in a second quadrant of the circumference of the person's wrist, arm, or finger; and a third light emitter in the array can be located in a third quadrant of the circumference of the person's wrist, arm, or finger. In an example, a first light receiver in an array can be located in a first quadrant of the circumference of the person's wrist, arm, or finger; a second light receiver in the array can be located in a second quadrant of the circumference of the person's wrist, arm, or finger; and a third light receiver in the array can be located in a third quadrant of the circumference of the person's wrist, arm, or finger.

In an example, the circumferential (e.g. polar, compass, or clock-hour) location of a light emitter on the circumference of the person's wrist, finger, or arm can be automatically changed over time by the device. In an example, the circumferential (e.g. polar, compass, or clock-hour) location of a light emitter on the circumference of the person's wrist, finger, or arm can be automatically shifted and/or moved by the device. In an example, a device can have an arcuate channel, groove, or track around at least part of its circumference, along which a light emitter can be automatically moved (e.g. rotated) by an actuator in the device. In an example, a light emitter on a person's wrist, finger, or arm can be automatically moved (e.g. rotated) by the device from one circumferential quadrant of the person's wrist, finger, or arm to another circumferential quadrant of the person's wrist, finger, or arm. In an example, a light emitter can be moved (e.g. rotated) around at least half of the circumference of the device. In an example, the circumferential location of a light emitter on the circumference of the person's wrist, finger, or arm can be automatically oscillated by the device.

In an example, the circumferential (e.g. polar, compass, or clock-hour) location of a light receiver on the circumference of the person's wrist, finger, or arm can be automatically changed over time by the device. In an example, the circumferential (e.g. polar, compass, or clock-hour) location of a light receiver on the circumference of the person's wrist, finger, or arm can be automatically shifted and/or moved by the device. In an example, a device can have an arcuate channel, groove, or track around at least part of its circumference, along which a light receiver can be automatically moved (e.g. rotated) by an actuator in the device. In an example, a light receiver on a person's wrist, finger, or arm can be automatically moved (e.g. rotated) by the device from one circumferential quadrant of the person's wrist, finger, or arm to another circumferential quadrant of the person's wrist, finger, or arm. In an example, a light receiver can be moved (e.g. rotated) around at least half of the circumference of the device. In an example, the circumferential location of a light receiver on the circumference of the person's wrist, finger, or arm can be automatically oscillated by the device.

In an example, there can be differences in the wavelengths, colors, and/or spectra of light energy emitted by light emitters at different locations in an array. In an example, a first light emitter in an array of sensors can emit light with a first wavelength, color, and/or spectrum and a second light emitter in the wearable ring of sensors can emit light with a second wavelength, color, and/or spectrum. In an example, there can be alternation between two light wavelengths and/or colors in an arcuate array of light emitters around (at least half of) the circumference of a person's wrist, finger, or arm. In an example, there can be a repeating sequence of two different light wavelengths and/or colors in an arcuate array of light emitters around (at least half of) the circumference of a person's wrist, finger, or arm. In an example, there can be a repeating sequence of three different light wavelengths and/or colors in an arcuate array of light emitters around (at least half of) the circumference of a person's wrist, finger, or arm.

In an example, there can be changes in the wavelength, color, and/or spectrum of light energy emitted by the same light emitter over time. In an example, a light emitter in an array of sensors can emit light with a first wavelength, color, and/or spectrum at a first time and emit light with a second wavelength, color, and/or spectrum at a second time. In an example, the wavelength, color, and/or spectrum of light energy emitted by a light emitter can be automatically oscillated by the device in order to scan body tissue at different depths and/or locations. In an example, the wavelength, color, and/or spectrum of light energy emitted by a light emitter can be automatically oscillated by the device in order to scan body tissue at different spectral wavelengths. In an example, the wavelength, color, and/or spectrum of light energy emitted by a light emitter can be automatically oscillated by the device in order to scan for different biometric parameters.

In an example, there can be differences in the angles and/or vectors along which beams of light are emitted toward a person's body by light emitters at different locations in an array. In an example, beams of light can be emitted from different light emitters along different angles and/or vectors in order to scan different tissue depths and/or regions. In an example, beams of light emitted from different light emitters can hit the surface of a person's wrist, finger, or arm at different angles and/or vectors in order to scan different tissue depths and/or regions. In an example, a first light emitter in an array can emit light along first angle and/or vector with respect to the person's body and a second light emitter in the array can emit light along a second angle and/or vector with respect to the person's body. In an example, the angle and/or vector of light emitted from a light emitter can be automatically changed over time by the device. In an example, the angle and/or vector of light emitted from a light emitter can be automatically changed over time by movement of a micromirror, microprism, or microlens. In an example, the angle and/or vector of light emitted from a light emitter can be automatically oscillated by the device.

In an example, there can be differences in the distance and/or pressure between different light emitters and a person's wrist, finger, or arm. In an example, there can be a first distance and/or pressure between a first light emitter in the array and a person's body surface and a second distance and/or pressure between a second light emitter in the array and the person's body surface. In an example, the distance and/or pressure between a light emitter relative to the person's body can be automatically changed over time by the device. In an example, the distance and/or pressure from a light emitter relative to the person's body can be automatically oscillated by the device.

In an example, an array of sensors can further comprise one or more motion sensors, wherein a motion sensor can further comprise an accelerometer and/or gyroscope. In an example, different light emitters in the array can be selectively activated to emit light at a given time based on data from the one or more motion sensors. In an example, different light emitters in the array can be selectively activated in order to maintain spectroscopic scanning of the same tissue region even when a device shifts and/or rotates. In an example, the circumferential locations of one or more light emitters in the array can be selectively moved based on data from the one or more motion sensors. In an example, the circumferential locations of one or more light emitters in the array can be selectively moved in order to maintain spectroscopic scanning of the same tissue region even when a device shifts and/or rotates. In an example, the angle and/or vector of a beam of light emitted from a light emitter can be automatically changed by the device based on data from the one or more motion sensors. In an example, when the wearable ring shifts and/or rotates on a person's wrist, arm, or finger, the device can automatically change the angle and/or vector of light beams emitted from one or more light emitters in order to maintain spectroscopic scanning of the same tissue region even when a device shifts and/or rotates.

In an example, an array of sensors can further comprise one or more motion sensors, wherein a motion sensor can further comprise an accelerometer and/or gyroscope. In an example, the circumferential locations of one or more light receivers in the array can be selectively moved based on data from the one or more motion sensors. In an example, the circumferential locations of one or more light receivers in the array can be selectively moved in order to maintain spectroscopic scanning of the same tissue region even when a device shifts and/or rotates.

In an example, an array of sensors can further comprise one or more distance and/or pressure sensors. In an example, different light emitters in the array can be selectively activated to emit light at a given time based on data from the one or more distance and/or pressure sensors. In an example, the circumferential locations of one or more light emitters in the array can be selectively moved based on data from the one or more distance and/or pressure sensors. In an example, the angle and/or vector of a beam of light emitted from a light emitter can be automatically changed by the device based on data from the one or more distance and/or pressure sensors.

In an example, light emitters can be LEDs. In an example, light emitters can be lasers. In an example, different light emitters in the array can emit light energy with different wavelengths and/or spectra. In an example, different light emitters in the array can emit light energy at different angles and/or vectors with respect to the surface of the person's body. In an example, different light emitters in the array can emit light energy at different times.

In an example, a device can have (four or more) inflatable compartments or protrusions around the inner circumference of the ring. In an example, light emitters can be located on the inflatable compartments or protrusions. In an example, light receivers can be located on the inflatable compartments or protrusions. In an example, a device can have (four or more) compressible protrusions around the inner circumference of the ring. In an example, light emitters can be located on the compressible protrusions. In an example, light receivers can be located on the compressible protrusions. In an example, a device can have (four or more) flexible, elastic, and/or compressible undulations around the circumference of the ring. In an example, light emitters can be located on inward curving portions of the undulations. In an example, light receivers can be located on inward curving portions of the undulations.

In an example, this device can further comprise a data processor and a data transmitter/receiver. In an example, this device can further comprise a power source or transducer which generates power from human movement and/or thermal energy. In an example, this device can further comprise a display, such as a display screen. In an example, this device can further comprise one or more compressible and/or elastomeric light shields between light emitters and light receivers. In an example, this device can further comprise one or more motion sensors (such as accelerometers and/or gyroscopes). In an example, this device can further comprise one or more pressure sensors. In an example, pressure sensors can be distributed around the circumference of the body-facing (inward) surface of the ring in order to measure greater and lesser of contact between the device and the person's wrist, finger, or arm.

In various examples, this device can further comprise one or more components selected from the group consisting of: data processing member, data transmitting member, data receiving member, power source, and energy harvester. In various examples, this device can communicate with a handheld electronic device, a different wearable technology device, an array of wearable sensors, a communication network tower, a satellite, a home control system, and/or an implantable medical device. In various examples, this device can further comprise one or more components selected from the group consisting of: one or more LEDs; one or more coherent light emitters or projectors; one or more infrared light emitters or projectors; one or more sound-emitting members; one or more tactile-sensation-creating members; one or more neurostimulators, myostimulators, or other electromagnetic energy emitters; one or more hardware buttons, knobs, or keys, a virtual projected keypad; a gesture-recognition interface; a speech-recognition interface, and an eye-gaze-tracking interface.

In an example, a wearable computing device for the wrist and/or forearm can comprise: a display; a motion sensor; and an attachment member which is configured to be fastened around the wrist and/or forearm, wherein the device has a first configuration in which the attachment member has a first degree of contraction in order to fit in a relatively loose manner around the wrist and/or forearm and a second configuration in which the attachment member has a second degree of contraction in order to fit in a relatively snug manner around the wrist and/or forearm, and wherein the device is automatically and temporarily changed from the first configuration to the second configuration based on information from the motion sensor. In an example, the motion sensor can include one or more components selected from the group consisting of: accelerometer; gyroscope; and inclinometer. In an example, the attachment member can contract it is when exposed to electrical current. In an example, the device can further comprise an electromagnetic actuator which contracts the attachment member.

In an example, a wearable computing device for the wrist and/or forearm can comprise: a display; a optical sensor; and an attachment member which is configured to be fastened around the wrist and/or forearm, wherein the device has a first configuration in which the attachment member has a first degree of contraction in order to fit in a relatively loose manner around the wrist and/or forearm and a second configuration in which the attachment member has a second degree of contraction in order to fit in a relatively snug manner around the wrist and/or forearm, and wherein the device is automatically and temporarily changed from the first configuration to the second configuration based on information from the optical sensor. In an example, the optical sensor can include one or more components selected from the group consisting of: spectroscopic sensor, light-spectrum-analyzing sensor, optoelectronic sensor, PPG sensor, and infrared light sensor. In an example, the attachment member can contract it is when exposed to electrical current. In an example, the device can further comprise an electromagnetic actuator which contracts the attachment member.

In an example, a wearable computing device for the wrist and/or forearm can comprise: a display; a compression sensor; and an attachment member which is configured to be fastened around the wrist and/or forearm, wherein the device has a first configuration in which the attachment member has a first degree of contraction in order to fit in a relatively loose manner around the wrist and/or forearm and a second configuration in which the attachment member has a second degree of contraction in order to fit in a relatively snug manner around the wrist and/or forearm, and wherein the device is automatically and temporarily changed from the first configuration to the second configuration based on information from the compression sensor. In an example, the compression sensor can include one or more components selected from the group consisting of: pressure sensor, strain gauge, and piezoelectric sensor. In an example, the attachment member can contract it is when exposed to electrical current. In an example, the device can further comprise an electromagnetic actuator which contracts the attachment member. Relevant variations which are discussed in priority-linked disclosures can also be applied to the example shown in FIG. 1.

I claim:

1. A wearable computing device for the wrist and/or forearm comprising:
a display;
a motion sensor; and
an attachment member which is configured to be fastened around a wrist and/or forearm, wherein the device has a first configuration in which the attachment member has a first degree of contraction in order to fit in a relatively loose manner around the wrist and/or forearm and a second configuration in which the attachment member has a second degree of contraction in order to fit in a relatively snug manner around the wrist and/or forearm, and wherein the device transitions from the first configuration to the second configuration based on information from the motion sensor.

2. The device in claim 1 wherein the motion sensor is an accelerometer.

3. The device in claim 1 wherein the motion sensor is a gyroscope.

4. The device in claim 1 wherein the motion sensor is an inclinometer.

5. The device in claim 1 wherein the attachment member contracts when exposed to electrical current.

6. The device in claim 1 wherein the device further comprises an actuator which contracts the attachment member.

7. A wearable computing device for the wrist and/or forearm comprising:
a display;
an optical sensor; and
an attachment member which is configured to be fastened around a wrist and/or forearm, wherein the device has a first configuration in which the attachment member has a first degree of contraction in order to fit in a relatively loose manner around the wrist and/or forearm and a second configuration in which the attachment member has a second degree of contraction in order to fit in a relatively snug manner around the wrist and/or forearm, and wherein the device transitions from the first configuration to the second configuration based on information from the optical sensor.

8. The device in claim 7 wherein the optical sensor is a spectroscopic sensor and/or light-spectrum-analyzing sensor.

9. The device in claim 7 wherein the optical sensor is an optoelectronic sensor.

10. The device in claim 7 wherein the optical sensor is an infrared light sensor.

11. The device in claim 7 wherein the attachment member contracts when exposed to electrical current.

12. The device in claim 7 wherein the device further comprises an actuator which contracts the attachment member.

13. A wearable computing device for the wrist and/or forearm comprising:
a display;
a compression sensor; and
an attachment member which is configured to be fastened around a wrist and/or forearm, wherein the device has a first configuration in which the attachment member has a first degree of contraction in order to fit in a relatively loose manner around the wrist and/or forearm and a second configuration in which the attachment member has a second degree of contraction in order to fit in a relatively snug manner around the wrist and/or forearm, and wherein the device transitions from the first configuration to the second configuration based on information from the compression sensor.

14. The device in claim 13 wherein the compression sensor is a pressure sensor.

15. The device in claim 13 wherein the compression sensor is a strain gauge.

16. The device in claim 13 wherein the compression sensor is a piezoelectric sensor.

17. The device in claim 13 wherein the attachment member contracts when exposed to electrical current.

18. The device in claim 13 wherein the device further comprises an actuator which contracts the attachment member.
